(19) United States
(12) Patent Application Publication
(10) Pub. No.: US 2023/0207101 A1
(43) Pub. Date: Jun. 29, 2023

(54) METHOD AND SYSTEM FOR IMPROVING A MEAL

(71) Applicant: DayTwo Ltd., Tel Aviv (IL)

(72) Inventors: Yaron VAZANA, Tel Aviv (IL); Shelly ABADI, Kfar Saba (IL); Uri ZONENS, Tel Aviv (IL); Adi LEV, Ganei Hadar (IL); Elinor AXELROD AVRAHAM, Hod Hasharon (IL); Noa GREEN, Givatayim (IL)

(73) Assignee: DayTwo Ltd., Tel Aviv (IL)

(21) Appl. No.: 18/090,558

(22) Filed: Dec. 29, 2022

Related U.S. Application Data
(60) Provisional application No. 63/294,395, filed on Dec. 29, 2021.

(51) Int. Cl. G16H 20/60; G16H 40/67; G16H 10/60

(52) U.S. Cl. CPC G16H 20/60; G16H 40/67; G16H 10/60

(57) ABSTRACT

A method of providing a subject with a personalized diet comprises: receiving from a graphical user interface (GUI) at a remote location a list of food items defining a meal, obtaining a set of subject descriptor features specific to the subject, and constructing a meal vector based on the list. Similarities between the meal vector and vectors in a database of food vectors are calculated, and at least one database vector is selected based on the similarities and the set of subject descriptor features. Based on the selected database vector(s), an identification of at least one food item is transmitted to the remote location for displaying the identification on the GUI as one or more food item(s) that is an addition to the meal.

### RELATED APPLICATION(S)

This application claims the benefit of priority under 35 USC §119(e) of U.S. Provisional Patent Application No. 63/294,395 filed on Dec. 29, 2021, the contents of which are incorporated by reference as if fully set forth herein in their entirety.

### FIELD AND BACKGROUND OF THE INVENTION

The present invention, in some embodiments thereof, relates to personalized diet and, more particularly, but not exclusively, to a method and system for improving a meal of an individual.

Obesity and Type 2 diabetes (T2D) mellitus incidence in the U.S. has increased dramatically over the past decades. Incidence of Type 2 diabetes, and co-morbidities, such as cardiovascular disease, has increased in direct relation to obesity. The epidemic of these chronic diseases has led to an emphasis on dietary management. Elevated blood glucose levels are associated with a higher risk of developing T2D and other metabolic diseases [1-3]. The prevalence of hyperglycemia, prediabetes, and T2D has dramatically increased over the last few years [4].

Several computerized dietary techniques have been proposed. International Publication No. W02002100266 discloses a dietary technique which employs a database of reference human factors. A computer receives user data on the human factors and predicts a selected characteristic of blood of the user dependent upon user data and measured blood characteristics, to generate a prediction model. The prediction model is then interrogated to generate and display a predicted blood characteristic dependent upon input human factor data. Also disclosed is the use of a database which includes a prediction model on a selected blood characteristic as a function of a human factor.

International Publication No. W02015/166489, the contents of which are hereby incorporated by reference, discloses a method of predicting a response of a subject to food, by selecting a food to which a response of the subject is unknown, and using information describing the subject and knowledge of responses of other subjects to foods to estimate the response of the subject to the selected food.

### SUMMARY OF THE INVENTION

According to an aspect of some embodiments of the present invention there is provided a method of providing a subject with a personalized diet. The method comprises: receiving from a graphical user interface (GUI) at a remote location a list of food items defining a meal, obtaining a set of subject descriptor features specific to the subject, and constructing a meal vector based on the list. A database storing a plurality of database vectors, each defining a food item, is accessed and similarities between the meal vector and the database vectors are calculated. The method selects, based on the similarities and the set of features, at least one database vector. Based on the selected database vector(s), an identification of at least one food item is transmitted to the remote location for displaying the identification on the GUI as one or more food item(s) that is/are an addition to the meal. The food item(s) that are displayed as addition(s) to the meal optionally and preferably serve as meal improvers.

According to some embodiments of the invention the method comprises representing each food item of the meal as a food vector, thereby providing a list of food vectors, wherein the constructing the meal vector is based on the list of food vectors. According to some embodiments of the invention the meal vector is constructed by averaging over the list of food vectors.

According to some embodiments of the invention the representaction of each food item of the meal as a food vector is by a look-up table associating food items with food vectors.

According to some embodiments of the invention the method comprises obtaining a cohort to which the subject belongs, wherein the selection of the database vector(s) is based also on the cohort.

According to some embodiments of the invention the method comprises accessing a computer-readable medium storing a library of look-up tables, each corresponding to a different cohort and each associating food items with food vectors, and selecting from the library a look-up table corresponding to the obtained cohort, wherein the representing is by the selected look-up table.

According to some embodiments of the invention the method comprises accessing a computer-readable medium storing a machine learning procedure trained to classify meal vectors into one of a plurality of predefined meal classifications, feeding the machine learning procedure with the constructed meal vector, and receiving from the procedure an output indicative of a classification of the constructed meal vector, wherein the selecting is based also on the classification.

According to some embodiments of the invention the plurality of predefined meal classifications comprises a first meal classification defined as a primary meal, a second meal classification defined as a secondary meal, and a third meal classification defined as a snack.

According to some embodiments of the invention the selection of database vector comprises selecting k database vectors which are most similar to the meal vector, where k is a positive integer. According to some embodiments of the invention the method comprises accessing a computer-readable medium storing a machine learning procedure trained to classify meal vectors into one of a plurality of predefined meal classifications, feeding the machine learning procedure with the constructed meal vector, receiving from the procedure an output indicative of a classification of the constructed meal vector, and filtering the k database vectors based on the classification.

According to some embodiments of the invention the computer-readable medium stores a library of machine learning procedures each corresponding to a different cohort, wherein the method comprises selecting from the library a machine learning procedure corresponding to the obtained cohort, and wherein the feeding and the receiving is with respect to the selected procedure.

According to some embodiments of the invention a plurality of database vectors are selected, and the method comprises accessing a computer-readable medium storing a machine learning procedure trained to rank food items based on subject descriptor features, feeding the machine learning procedure with the set of subject descriptor features and each of the database vectors, and receiving from the procedure an output indicative of rankings of the database vectors for the subject, wherein the transmission of the identification of the at least one food item is based on the rankings.

According to some embodiments of the invention the method comprises decomposing each food item of the meal into a plurality of food descriptors, and feeding the machine learning procedure also with the plurality of food descriptors.

According to some embodiments of the invention at least one of the food descriptors is selected from the group consisting of a carbohydrate content of the food item, a fat content of the food item, a dietary-fiber content of the food item, a caloric value content of the food item, a protein content of the food item, a sugar content of the food item, a water content of the food item, and a total weight of the food item.

According to some embodiments of the invention a plurality of database vectors are selected, and the method comprises accessing a computer-readable medium storing a machine learning procedure trained to predict responses to food items based on subject descriptor features, feeding the machine learning procedure with the set of subject descriptor features and each of the database vectors, and receiving from the procedure an output indicative of predictions of responses of the subject to food items described by the database vectors, wherein the transmission of the identification of the at least one food item is based on the predictions.

According to an aspect of some embodiments of the present invention there is provided a computer software product, comprises a computer-readable medium in which program instructions are stored, which instructions, when read by a data processor, cause the data processor to execute the method as delineated above and optionally and preferably as further detailed below.

According to an aspect of some embodiments of the present invention there is provided a server system for providing a subject with a personalized diet, the server system comprises: a transceiver arranged to receive and transmit information on a communication network; and a processor arranged to communicate with the transceiver, and execute the method as delineated above and optionally and preferably as further detailed below.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart diagram of a method suitable for providing a subject with a personalized diet, according to some embodiments of the present invention;

FIG. 2 is a schematic illustration of a library suitable representing food items as food vectors, according to some embodiments of the present invention;

FIG. 3 is a schematic illustration of a client-server configuration which can be used according to some embodiments of the present invention for providing a subject with a personalized diet, according to some embodiments of the present invention.

FIG. 4 is a schematic diagram of a method used in experiments performed according to some embodiments of the present invention;

FIG. 5 is a schematic illustration of a network architecture used in experiments performed according to some embodiments of the present invention;

FIG. 6 is a schematic illustration demonstrating similarities between two meals, between two food items, and between a meal and a food item, according to some embodiments of the present invention;

FIG. 7 shows feature importance for three meal classifiers, according to some embodiments of the present invention;

FIG. 8 is a schematic illustration of an architecture of a ranking procedure according to some embodiments of the present invention;

FIGS. 9A-D show results of a ranking model used in experiments performed according to some embodiments of the present invention; and

FIGS. 10A-F are histograms showing results obtained in experiments performed according to some embodiments of the present invention.

### DESCRIPTION OF SPECIFIC EMBODIMENTS OF THE INVENTION

The present invention, in some embodiments thereof, relates to personalized diet and, more particularly, but not exclusively, to a method and system for improving a meal of an individual.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

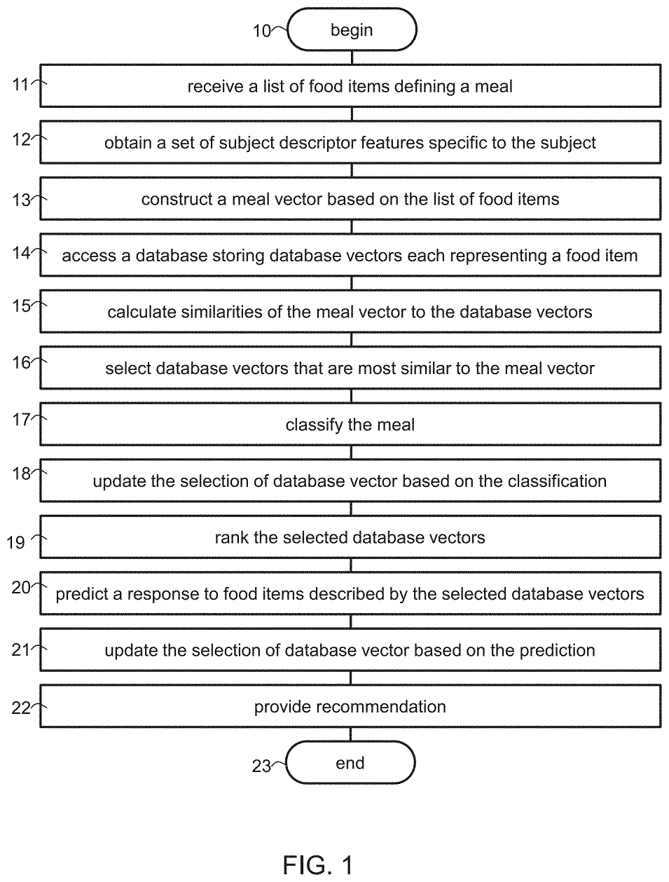

Referring now to the drawings, FIG. 1 is a flowchart diagram of a method suitable for providing a subject with a personalized diet, according to various exemplary embodiments of the present invention. It is to be understood that, unless otherwise defined, the operations described hereinbelow can be executed either contemporaneously or sequentially in many combinations or orders of execution. Specifically, the ordering of the flowchart diagrams is not to be considered as limiting. For example, two or more operations, appearing in the following description or in the flowchart diagrams in a particular order, can be executed in a different order (e.g., a reverse order) or substantially contemporaneously. Additionally, several operations described below are optional and may not be executed.

At least part of the operations described herein can be implemented by a data processing system, e.g., a dedicated circuitry or a general purpose processor, configured for executing the operations described below. At least part of the operations can be implemented by a cloud-computing facility at a remote location.

Computer programs implementing the method of the present embodiments can commonly be distributed to users by a communication network or on a distribution medium such as, but not limited to, a floppy disk, a CD-ROM, a flash memory device and a portable hard drive. From the communication network or distribution medium, the computer programs can be copied to a hard disk or a similar intermediate storage medium. The computer programs can be run by loading the code instructions either from their distribution medium or their intermediate storage medium into the execution memory of the computer, configuring the computer to act in accordance with the method of this invention. During operation, the computer can store in a memory data structures or values obtained by intermediate calculations and pull these data structures or values for use in subsequent operation. All these operations are well-known to those skilled in the art of computer systems.

Processing operations described herein may be performed by means of processer circuit, such as a DSP, microcontroller, FPGA, ASIC, etc., or any other conventional and/or dedicated computing system.

The method of the present embodiments can be embodied in many forms. For example, it can be embodied in on a tangible medium such as a computer for performing the method operations. It can be embodied on a computer-readable medium, comprising computer-readable instructions for carrying out the method operations. It can also be embodied in electronic device having digital computer capabilities arranged to run the computer program on the tangible medium or execute the instruction on a computer-readable medium.

In various exemplary embodiments of the invention the method is executed on behalf of a service provider, such as, but not limited to, a provider of personalized diet recommendations. In these embodiments of the invention, the subject is a member of a group of identified subjects, each having an existing user account with the service provider, and the subject is requested to log into their user account to allow the method to provide the service to the identified subject.

The method begins at 10 and optionally and preferably continues to 11 at which a list of food items is received. The received list is collectively referred to as a meal. Typically, the list of food items that defines the meal is selected by a subject at a remote location. For example, the subject can operate a graphical user interface (GUI) in order to select the food items. The GUI can be a web page or a GUI of an app of a mobile device (e.g., smartphone or a tablet or the like). Preferably, the GUI includes a display of multiplicity of food items, and a respective multiplicity of GUI selection controls allowing the subject to select individual food items one by one or in groups to define the meal. Controls activated by the subject can be communicated from the GUI to a data processor of a computer or the mobile device, and the data processor can process the controls, identify which food items have been selected by the subject, and transmit data pertaining to these food items over a communication network to a server computer or a cloud computing facility, remote to the subject.

When the subject has an existing user account with the service provider, the method can identify the subject based on the user account and associate the received meal with the identified subject.

The method continues to 12 at which a set of subject descriptor features which are specific to the subject are obtained. Typically, these descriptor features are read from a computer-readable medium storing a set of descriptor features for each of a multiplicity of identified subjects having user account with the service provider. Alternatively, the descriptor features can be uploaded from the remote location as a datafile which is associated with the received meal.

The descriptor features typically also include general information such as gender, age, and preferably also a cohort to which the subject belongs. The cohort is typically a demographic cohort and is indicative of demographic characteristics of the subject, including, without limitation, a geographic origin of the subject, a geographic region at which the subject currently lives, cultural mores, and the like. The descriptor features can additionally include at least one of: a microbiome profile or a partial microbiome profile of the subject obtained by analyzing a stool sample of the subject, blood chemistry or partial blood chemistry of the subject [e.g., results of one or more tests such as, but not limited to, a triglyceride test, a glycosylated hemoglobin (HbAlc) test, a connecting peptide (C-peptide) test, a fasting plasma glucose (FPG) test, an oral glucose tolerance test (OGTT), and a casual plasma glucose test], genetic profile or partial genetic profile of the subject, metabolomic data associated with the subject, a medical condition of the subject. The descriptor features can optionally and preferably also include size parameters such as height, weight, and/or BMI. In experiments performed by the inventors the following descriptor features were employed: cohort, gender, age, height, weight, BMI, FPG test results, triglyceride test results, HbAlc test results, and microbiome related features extracted from a stool sample of the subject.

The method optionally and preferably continues to 13 at which a meal vector is constructed based on the list of food items obtained at 11. In embodiments in which the method obtains a cohort to which the subject belongs, the meal vector that is constructed is specific both to the meal and to the cohort, so that when individual subjects belonging to different cohorts select the same meal, the meal vector that are constructed to those individual subjects may differ. Typically, each food item of the meal is represented as a food vector, and the meal vector is constructed by based on these food vectors. For example, the meal vector can be defined as average of the food vectors of all the food items of the meal in a vector space in which the food vectors are embedded. Such an average can in some embodiments of the present invention be calculated as a component-wise mean of the food vectors.

Food items can be represented as food vectors within a predefined vector space. Practically, this can be done by accessing a computer-readable medium storing a look-up table that is prepared in advance, and that associates food items with food vectors. Each entry of this look-up table includes a food item and a plurality of components that define a food vector associated with the food item. The look-up table can include an entry for each of the multiplicity of food items from which the subject selects the list of food items that defines the meal, so that each of meal's food items can be associated by a food vector using the look-up table. It is appreciated that the multiplicity of food items displayed by the GUI can be updated from time to time, for example, based on feedback received from other subjects that have user account with the service provider. In this case, the look-up table can also be updated accordingly.

In some embodiments of the present invention all the vectors in the look-up table have the same dimensionality. Preferably, the vectors in the look-up table have at least 10 or at least 20 or at least 30 dimensions, e.g., about 40 dimensions or more. Herein, the dimensionality of the vector is defined as the number of components that form the vector. In experiments performed by the present inventors, food items have been represented by vectors having 40 dimensions.

The look-up table optionally and preferably assigns to food items that are similar to each other, food vectors that are similar to each other in the vector space at which they are embedded. The similarity between vectors can be defined according to any vector space metric, such as, but not limited to, Euclidian distance, cosine similarity, p-norm of a difference between vectors, and the like.

According to some embodiments of the present invention the look-up table can thus be updated even without updating the multiplicity of food items displayed by the GUI. Such an update is optionally and preferably executed when a sufficient amount of data arrives from subjects having user account with the service provider, and/or periodically. For example, suppose that many subjects select at 11 lists of food that are not necessarily identical but nevertheless include a particular subset of food items. In this case there is a high likelihood that the food items in this subset are similar top each other, and so the look-up table is preferably updated to ensure that the similarity of the food vectors that are assigned to the food items in this subset is sufficiently high.

The look-up table can be constructed by means of natural language processing (NLP) techniques, considering each food item as a natural language word, and pairs of similar food items as natural language words that are similar in their meaning. Such NLP technique can be applied each time the multiplicity of food items is updated. Representative examples of NLP techniques suitable for the present embodiments include, without limitation, neural networks, dimensionality reduction, probabilistic models, explainable knowledge base models, and the like.

In some embodiments an NLP technique assigns vectors based on pairwise similarities among the food items. For example, each of the food vectors to be included in the look-up table can be represented as a unique descriptor vector in a descriptor vector space. Conveniently, such a unique descriptor vector can be a one-hot encoded vector (e.g., a vector in which all but one of its elements are zero, wherein the position with the vector of the non-zero element is unique for each food item), but any other formalism that ensures unique assignment can be employed. Then for each pair of food items that are considered to be similar to each other, a machine learning procedure can be trained to predict a descriptor vector of each food item of the pair, given the other food item of the pair. Once the machine learning procedure is trained using all the food items to be included in the look-up table, the weights of the machine learning procedure can be used to construct a food vector for each food item. Since the training is executed pairwise among food items considered to be similar, this ensures that similar food items are assigned with similar food vectors. Typically, learned weights of the machine learning procedure are arranged as matrix-elements in an embedding matrix W, and the food vector for each food item can be computed by multiplying the descriptor vector of this food item by the embedding matrix W. A more detailed example of a neural network based technique suitable for generating a look-up table is provided in the Examples section that follows.

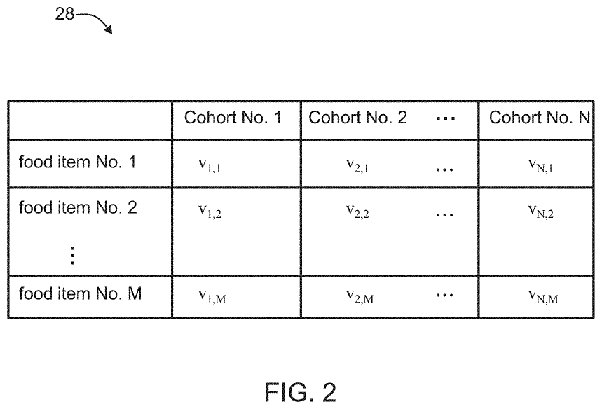

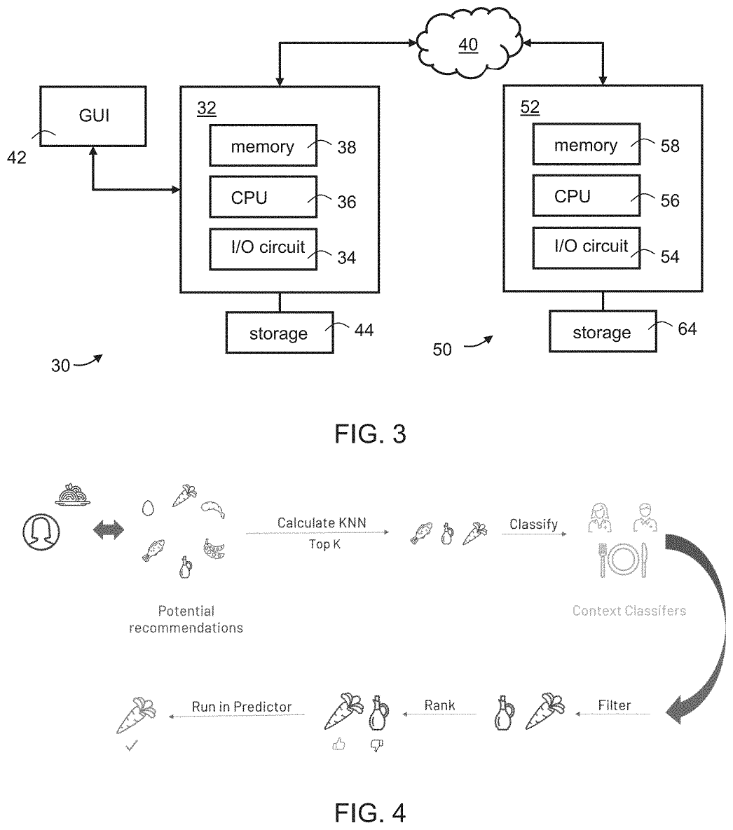

In embodiments in which the method obtains a cohort to which the subject belongs, the specificity of the meal vector to the cohort can be ensured by selecting a look-up table that has been prepared in advance for the specific cohort and using this selected table for representing the food items as food vectors. Specifically, the method can access a computer-readable medium storing a library of look-up tables, each corresponding to a different cohort and each associating food items with food vectors. A representative example of such a library 28 is illustrated in FIG. 2. The left column in FIG. 2 includes all food items (enumerated 1 through M) from which the subject can select the list which defines the meal. Each of the other columns defines a different vector space for each of a plurality of different cohorts (enumerated 1 through N). For each vector space, each food item of the left column is associated with a vector. Thus, left column forms with each of the other columns a look-up table that is specific to the respective cohort. The vector that is associated by the ith look-up table (corresponding to the ith cohort) to the jth food item is denoted in FIG. 2 as vᵢⱼ.

Referring again to FIG. 1, at 14 the method accesses a database storing a plurality of database vectors, each representing a food item. The database preferably stores database vectors that are associated with food items in the same way that the food items of the meal are associated with food vectors, e.g., using the aforementioned look-up table(s). Thus, in various exemplary embodiments of the invention the database vectors are embedded in the same vector space as the food vectors. Preferably, all the database vectors have the same dimensionality. Preferably, the dimensionality of the database vectors is the same as the dimensionality of the food vectors defined using the look-up table(s). The database need not to include database vectors for all the food items in the multiplicity of food items from which the subject selects the meal (although such embodiments are also contemplated). Preferably, the database includes database vectors which describe food items that are generally considered as nutritionally beneficial to individuals in the general population. The database vectors can describe food items that are a subset of the multiplicity of food items from which the subject selects the meal (e.g., those food items that are considered nutritionally beneficial), or they can describe food items that absent from this multiplicity of food items, or, they can include both food items that are in the multiplicity of food items and both food items that are absent from this multiplicity of food items.

The size of the database accessed at 14 is typically from a few tens (e.g., from about 50 or from about 60 or from about 70 or from about 80 or from about 90) of database vectors to one or more hundred (e.g., to about 100 or to about 150 or to about 200 or to about 250 or to about 300) database vectors. In experiments performed by the inventors a database of about 200 database vectors was used.

The method proceeds to 15 at which similarities of the meal vector to the database vectors is calculated. Preferably, the entire database is traversed and the similarity to the meal vector is calculated separately for each database vector. Alternatively, only a portion of the database is traversed in which case the similarity to the meal vector is calculated only for the database vectors in this portion of the database. The similarity can be calculated using any vector space metric, including, without limitation, Euclidian distance, cosine similarity, p-norm of a difference between vectors, and the like. It is appreciated that the relation between the similarity and the metric depends on the selected metric. Specifically, when the metric is cosine similarity, higher values obtained for the metric are indicative of higher similarity, and when the metric is an Euclidian distance or a p-norm of a difference between vectors, lower values obtained for the metric are indicative of higher similarity. Based on the calculated similarities the method can provides the subject with a recommendation how to improve the meal. Broadly speaking, database vectors that are most similar to the meal vector are referred to as the “nearest neighbors” of the meal vector, and are used according to some embodiments of the present invention for providing the recommendation. For example, the method can provide the subject with a recommendation to include in the meal, not only the food items received at 11, but rather one or more food items that are described by nearest neighbors of the meal vector. A further processing, however, is optionally and preferably, but not necessarily, also employed before providing such a recommendation to the subject.

Hence, in some optional embodiments of the present invention, the method proceeds to 16 at which k nearest neighbors (KNN) are selected from the database, where k is a positive integer. Typical value for k is from about 10 to about 100. The KNN are the k database vectors that are most similar to meal vector in terms of the similarities calculated at 15. Optionally, the method continues to 17 at which the meal is classified into one of a plurality of predefined meal classifications. Such predefined meal classifications can include, for example, a first meal classification defined as a primary meal, a second meal classification defined as a secondary meal, and a third meal classification defined as a snack. The terms primary meal, secondary meal, and snacks, are typically defined based on the cohort, since different cultures attribute different classifications to food items.

The classification is based on the food items that define the meal. In some embodiments of the present invention the method accesses a computer-readable medium storing a machine learning procedure trained to classify meal vectors, feeds the machine learning procedure with the constructed meal vector, and receives from procedure an output indicative of the classification of the meal vector. The machine learning procedure can be of any type, such as, but not limited to, SVN, logistic regression, random forest, and XGboost, and the like. In various exemplary embodiments of the invention the machine learning procedure is trained to classify each meal vector also based on the cohort to which the subject belongs. Thus, in some embodiments of the present invention the computer-readable medium stores a library of machine learning procedures, each corresponding to a different cohort, and the method selects from this library a machine learning procedure corresponding to the cohort to which the subject belongs.

Following the classification 17, the method can proceed to 18 at which the KNN are filtered based on the classification. Specifically, the method can apply the classification procedure also to each database vector of the KNN, and then discard from consideration all the database vectors of the KNN which were not classified into the same classification as the meal.

It is appreciated that the classification procedure can also be applied to all the vectors of the database. Such operation can be executed in advance, so as to provide a plurality of databases, each corresponding to a different meal classification. In these embodiments, operation 17 optionally and preferably precedes operation 16, and operation 16 is executed only with respect top the database that shares the same classification as the meal.

The classification procedure that is applied to the database vectors (either to the entire database or only to the KNN) can be the same as the classification procedure that is applied to the meal vector.

In some embodiments of the present invention the method proceeds to 19 at which the selected (and optionally and preferably filtered) database vectors are ranked. Preferably, the ranking is specific to the subject, according to the descriptor features obtained at 12, and is executed by a machine learning procedure. Thus, in these embodiments, the method accesses a computer-readable medium storing a machine learning food ranking procedure that is trained to rank food items based on subject descriptor features. The machine learning food ranking procedure can be of any type, such as, but not limited to, a deep learning procedure, a convolutional neural network, and the like. A representative example of a machine learning food ranking procedure suitable for the present embodiments is described in greater detail in the Examples section that follows.

Once the food ranking procedure is obtained, the method feeds the machine learning procedure with one or more of the subject descriptor features and with each of the selected database vectors. Preferably, the method decomposes each food item of meal into a plurality of food descriptors, and feeds the food ranking procedure also with these food descriptors. The food descriptors for a particular food item of the meal can include, for example, at least one of: a carbohydrate content of the particular food item, a fat content of the particular food item, a dietary-fiber content of the particular food item, a caloric value content of the particular food item, a protein content of the particular food item, a sugar content of the particular food item, a water content of the particular food item, and a total weight of the particular food item. The method receives from the food ranking procedure an output indicative of rankings of the selected database vectors for the subject under analysis, and can use these ranking when providing the subject with the recommendation how to improve the meal. For example, the subject can be provided with a list of food items that can be added to the meal in an orderly manner, e.g., starting with the food item that is described by the highest ranked database vector and ending with the food item that is described by the lowest ranked database vector.

The method optionally and preferably continues to 20 at which the method predict the subject's response to the meal, when combined with one or more of the food items described by the selected (and optionally filtered) database vectors. The prediction is optionally and preferably by means of a machine learning procedure trained to predict responses to food items based on subject descriptor features. Such a machine learning procedure is known in the art and is described in International Publication No. W02015/166489, and in [Zeevi et al. Personalized nutrition by prediction of glycemic responses. Cell. 2015;163(5): 1079-1094. doi:10.1016/j.cell.2015.11.001] the contents of which are hereby incorporated by reference.

Typically, the machine learning procedure is trained to predict a glycemic response of the subject to food, but other types of responses, such as, but not limited to, changes in the levels of cholesterol, sodium, potassium, and/or calcium, are also contemplated.

Operation 20 is preferably executed for each of the selected database vectors separately. Specifically, the machine learning procedure is first fed with the meal and the food item described by one of database vectors, and an output pertaining to the predicted response is obtained. Then, the machine learning procedure is fed with the meal and the food item described by another one of database vectors, and an output pertaining to another predicted response is obtained. The process optionally and preferably continues until a predicted response is obtained for each of the database vectors.

The method can then proceed to 21 at which the selected database vectors are updated based on the prediction obtained at 20. Typically, the method can discard from the selected database vectors all database vectors for which the predicted response does not satisfy a predetermined criterion or set of criteria. For example, when the predicted response is a glycemic response, the method can discard database vectors for which the predicted change in the blood glucose level of the subject is above a predetermined threshold.

The method proceeds to 22 at which a recommendation is provided to the remote location. Typically, an identification of one or more food items that correspond(s) to the selected database vector(s), preferably following the updates at 18 and/or 21 and further preferably according to the ranking 19 is transmitted to the data processor at the remote for displaying the identification on the GUI as an addition to meal.

The method of the present embodiments can be executed according to some embodiments of the present invention by a server-client configuration, as will now be explained with reference to FIG. 2.

FIG. 2 illustrates a client computer 30 having a hardware processor 32, which typically comprises an input/output (I/O) circuit 34, a hardware central processing unit (CPU) 36 (e.g., a hardware microprocessor), and a hardware memory 38 which typically includes both volatile memory and non-volatile memory. CPU 36 is in communication with I/O circuit 34 and memory 38. Client computer 30 preferably comprises a user interface, e.g., a graphical user interface (GUI), 42 in communication with processor 32. I/O circuit 34 preferably communicates information in appropriately structured form to and from GUI 42. Also shown is a server computer 50 which can similarly include a hardware processor 52, an I/O circuit 54, a hardware CPU 56, a hardware memory 58. I/O circuits 34 and 54 of client 30 and server 50 computers preferable operate as transceivers that communicate information with each other via a wired or wireless communication. For example, client 30 and server 50 computers can communicate via a network 40, such as a local area network (LAN), a wide area network (WAN) or the Internet. Server computer 50 can be in some embodiments be a part of a cloud computing resource of a cloud computing facility in communication with client computer 30 over the network 40.

GUI 42 and processor 32 can be integrated together within the same housing or they can be separate units communicating with each other. GUI 42 can optionally and preferably be part of a system including a dedicated CPU and I/O circuits (not shown) to allow GUI 42 to communicate with processor 32. Processor 32 issues to GUI 42 graphical and textual output generated by CPU 36. Processor 32 also receives from GUI 42 signals pertaining to control commands generated by GUI 42 in response to user input. GUI 42 can be of any type known in the art, such as, but not limited to, a keyboard and a display, a touch screen, and the like. In preferred embodiments, GUI 42 is a GUI of a mobile device such as a smartphone, a tablet, a smartwatch and the like. When GUI 42 is a GUI of a mobile device, the CPU circuit of the mobile device can serve as processor 32 and can execute the method optionally and preferably by executing code instructions.

Client 30 and server 50 computers can further comprise one or more computer-readable storage media 44, 64, respectively. Media 44 and 64 are preferably non-transitory storage media storing computer code instructions for executing the method of the present embodiments, and processors 32 and 52 execute these code instructions. The code instructions can be run by loading the respective code instructions into the respective execution memories 38 and 58 of the respective processors 32 and 52. Storage media 64 preferably also store one or more databases including a database of psychologically annotated olfactory perception signatures as further detailed hereinabove.

In operation, processor 32 of client computer 30 displays on GUI 42 a multiplicity of food items, and a respective multiplicity of GUI selection controls allowing the subject to select individual food items one by one, or in groups, to define the meal. Processor 32 receives indications regarding the food items that are selected by the subject and transmits data pertaining to these selected food items to server computer 50 over network 40. Media 64 can store look-up table(s), machine learning procedure(s), and subject descriptor features as further detailed hereinabove. Server computer 50 can access media 64, and executes various operations of the method as described herein. Server computer 50 can also transmit to client computer 30 the food items that are described selected database vector(s) as further detailed hereinabove, client computer 30 can display this information on GUI 42.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### EXAMPLE 1

This Example describes an AI based food recommendation system for improving meals sugar scores, according to some embodiments of the present invention.

### Introduction

Elevated blood glucose levels are associated with a higher risk of developing serious health conditions such as type two diabetes (T2D) and additional metabolic diseases [1-3]. Known in the art is a technique for providing personalized dietary recommendations based on a sugar response machine learning model which predicts the user's glycemic response to different kinds of meals [5], and provides a score indicative of this predicted response. A major concern when asking people to eat meals above a certain score is the ongoing hassle in searching how to improve the meal's sugar score. In order to improve a meal score, a typical user will perform an exhaustive search by 1) thinking of possible food items which can be added to the meal—from culinarily perspective. 2) find the food items in the mobile app. 3) add the food items (one by one) to the existing meal and check whether it improves the overall meal sugar score. In case the score is not improved, the user will remove the food item and continue searching. The goal of the method presented in this Example is to avoid the search described above, by automating the entire recommendation process.

### Description of the Exemplified Method

The food domain is considered to be highly personalized, so one might prefer eating different meals depending on the emotional state, the location, the sensitivities to some ingredients, and more. This Example considers each cohort as a separate use case for the method where the outline remains the same but the data used at each step is taken from different sources. In addition, any required labeling task is also assigned to the appropriate domain experts with the relevant knowledge in the specific cohort. Both IL and US cohorts, are use cases in this Example that use a food database with approximately n=500k unique food items. A schematic diagram of the method of this Example is illustrated in FIG. 4.

The input to the method is a tuple of <user, meal>. The user is defined as a set of subject descriptor features which represents this specific user. In this Example, these features are divided into four groups 1) general information such as gender and age. 2) personal measurements such as height, weight and BMI. 3) blood tests results such as fasting glucose, triglycerides and HBAlC. 4) microbiome related features extracted from their stool sample. The meal is defined as a list of all the food items and is represented as a meal vector as described in Example 2, below. In addition, the method of this Example decomposes each food item into a into the following food descriptors: carbohydrates, fats, dietary-fibers, calories, protein, sugar, water and weight.

Initial food items list

A curated seed list of n food items (n was taken to be about 200 in this Example) served as a recommendations search space. The list was created by content experts with the notion that each item in the list can potentially improve some meal and the food item is considered a “healthy” item from the standpoint of being nutritionally beneficial. In order to generate the list, pre-trained food vectors which are described in Example 2, below, were used. First, a seed list with a few dozens of items was created, then, for each food item in the seed list the k=10 nearest neighbors were computed and added to the list. In this Example, this list is referred to as the search space list.

K nearest neighbors

Once the vectors of all the food items in the search space list, and the meal vector are defined, the top k=50 nearest neighbors for the meal vector (out of all the search space vectors) were computed, and the list was filtered to have only these top items.

Context classifiers

A set of machine learning classifiers which are referred to as context classifiers and which are described in Example 3, below, are applied to the vectors. The goal of these classifiers is to classify the meal into a few pre-defined contexts or classifications such as is_snack_meal, is_heavy_meal, is_light_meal. In this Example, each classifier included a binary classification model and there was no hierarchy between the classifiers, but other types of classifiers are also contemplated, with or without hierarchy. The search space list was then filtered according to the classification results. For example, for a given ice-cream meal (which is typically classified as a snack), all the items of the search space list which are not suitable to be added to ice-creams are removed. The classifiers serve for ensuring that the recommendations make sense culinarily. Thus, the list of the classifications is defined for each cohort separately in order to match each region's unique preferences.

Ranking

The list of filtered food items that represent the recommendations candidates, were then ranked in some order that takes into account the personalization and food preferences of each user. This was achieved by using a deep learning model which is described in Example 4, below. The model used four elements as inputs: the subject descriptor features, the meal vector, the meal nutrients and a single food item vector as described in Example 2. The output of the model was a positive score which represents how likely the food item is suitable to be added to the given meal for a specific user. In this Example this model was applied with the same user and meal for all the recommendation candidates and, and the recommendation candidates were sorted according to the output of the model. The model is optionally and preferably adapted from time to time based on feedback from the users so as to allow ranking the same food items in a different order according to different types of people. For example, this model may find yellow cheese to be more suitable for younger people and thus rank it higher for them.

Sugar response predictor

In order to make sure that the suggested items improve the sugar score of the existing meal, a sugar response predictor was applied. New meals, which were composed of the original meal together with each food item from the ranked list were simulated. In this Example, the food descriptors of the meal were summed together with the food descriptors of the food item, and were fed to the sugar response predictor. For each simulated meal, the predictor provided a sugar score. Only the food items which resulted in the simulated meal having a higher score than the original, were kept and returned to the user as recommendations.

### EXAMPLE 2

This Example describes a preferred technique for representing food items as high dimensional vectors. The technique is based on natural language processing and employs an unsupervised learning approach to directly learn a representation for food items data, using existing logged meals, as an element in using the food items themselves as inputs to machine learning models. In this Example, the word2vec neural network [7] was used.

Method

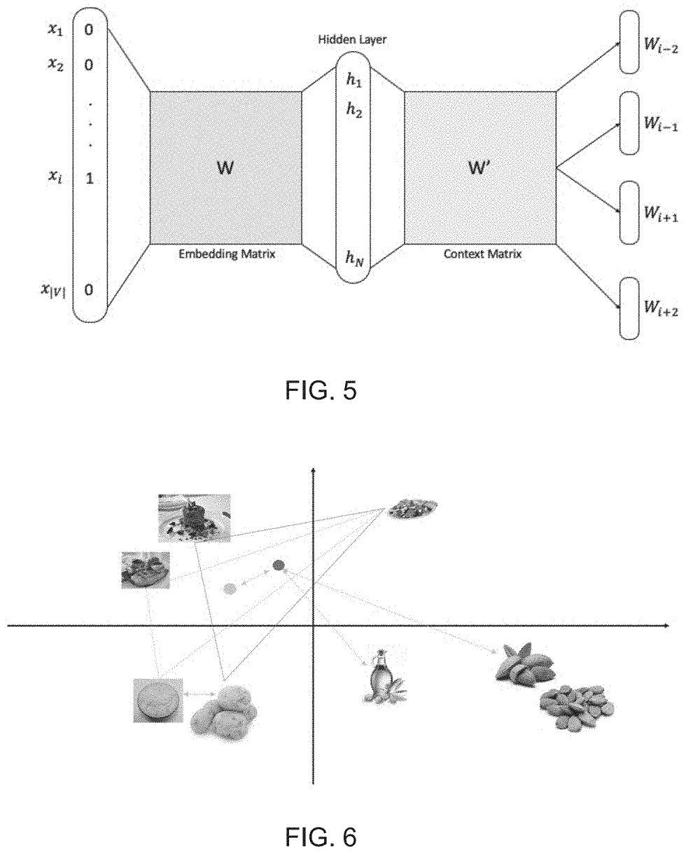

Given a set of n=6,086,051 logged meals, where each meal m is represented as a list of k unique food items ids m=(item 1, item 2, . . . , item k), a vector v of size d=40 is generated for each unique food item. The value d=40 was optimized using a bayesian optimization. Using the logged meals corpus, a total of 61,456 unique food items were obtained. Approximately, only 10% of all the food items were actually being used in a 6M meals corpus. In order to train the machine learning procedure (a word2vec neural network, in the present example), and obtain an embedding vector h for each food item, each meal was treated as a sentence of food items ids “id 1, id 2 , . . . , id k” , this allowed the use of existing word2vec libraries to train the network [8]. The skip-gram word2vec model was used with a window size of w equals to the maximum meal length in order to capture all the available food items pairs on all meals. FIG. 5 illustrates the word2vec network architecture, where the hidden layer is where each of the food vectors is computed, using learned weights arranged as matrix-elements in an embedding matrix

W.

Once the machine learning procedure was trained, the food vector h for each food item was computed by multiplying the descriptor vector x of this food item (a one-hot, it the present example) by the embedding matrix W.

Since the food domain is highly associated with geographic properties, the embeddings were trained for each cohort separately, including running hyper parameters optimization separately for each model.

Meals embeddings

The present inventors found that similar food items appear in similar meals, in the same manner in which word vectors are being learnt in the natural language processing domain. This way, a high dimensional space in which all the food items are embedded is obtained in a sense that similar food items have lower distance between their respective food vectors. In order to obtain a meal representation, all the food items vectors in the meal are averaged to provide a similar length vector which represents the entire meal. Specifically:

$$\vec{meal} = \frac{\sum \vec{{v}_{i}}}{n}$$

After obtaining a vector for each food item and for each meal, computing the similarities between them is done by using one of the well defined similarity metrics such as Cosine Similarity or Euclidean Distance. FIG. 6 demonstrates our ability to compute the similarities between two meals, between two food items, and between a meal and a food item, according to some embodiments of the present invention.

Embeddings quality

Different learning algorithms using different hyperparameters might encode different types of information which is not always aligned with the desired task. The inventors evaluated the quality of the learnt embedding before using them in downstream tasks. Existing schemes fall into two major categories: extrinsic and intrinsic evaluation. This example discusses the food embeddings as standalone vectors, so they were evaluated using precision@k metic, which is a well-known and which was and used for measuring the relevance of items in an output list. The evaluation process was done manually by content experts and included roughly n=100 food vectors with their k=10 nearest neighbors.

$$Precision @ k = \frac{number - of - recommended - items @ k - that - are - relevant}{number - of - recommended - items @ k}$$

In order to make sure that most of the food domain is covered, the test set was created manually and a few items from each category of <dairy, meat, fish, snacks, fruits, vegetable, drinks, condiments, chicken, sweets, pasta> were selected and validated.

### EXAMPLE 3

People can typically tolerate a recommendation system that provides them suggestions that they don not like to eat, but they would typically would be less tolerant for suggestions that break the culinary unwritten law to which they are familiar with. An example for a recommendation to which subjects may be less tolerant is a suggestion to add a beef meat to a chicken breast meal. Following is a description of a machine learning procedure that can be trained to classify meal vectors, according to some embodiments of the present invention. In this Example the meal is classified using a few binary classification models which are referred as context classifiers. The context classifiers determine together the type of the meal so as to formulate the underlying intent of the user. For example, a user which already selected a tomato and a cucumber is more likely to be eating a vegetable salad than eating some heavy lunch.

Method

The context classifiers were trained separately for each cohort, according to the specific needs of each geographical location. Also, the set of classifiers can be different for each cohort due to differences in eating habits each region has. In the present Example, the Israel cohort has three classifiers 1) heavy_classifier—this model detects meals that contain a main dish, e.g, when a user is eating a fish, chicken or a meat. 2) light_classifier—this model detects light meals such as a sandwich, mussily, salads, eggs etc. 3) snack classifier—this model detects snack meals such as ice-creams, cakes, cookies etc.

All the classifiers were trained in the same manner. Specifically, a bayesian optimization was applied on multiple machine learning model types (SVN, logistic regression, random forest and XGboost) together with their respective hyper-parameters. Each model received as an input following features: 1) the meal vector as described in Example 2. 2) one-hot-encoded meal categories—each food item in the meal can have one or more category, this information is encoded into a 1-hot vector. 3) ngrams features—unigrams and bigrams were extracted from all the names of the food items in the meal and were used as features. Each model generated an output in the form of a binary result of [0,1] according to the classifications decision. The models were applied in parallel and no model was using the results of the other models. Use of hierarchy among the models is also contemplated according to some embodiments of the present invention.

Results

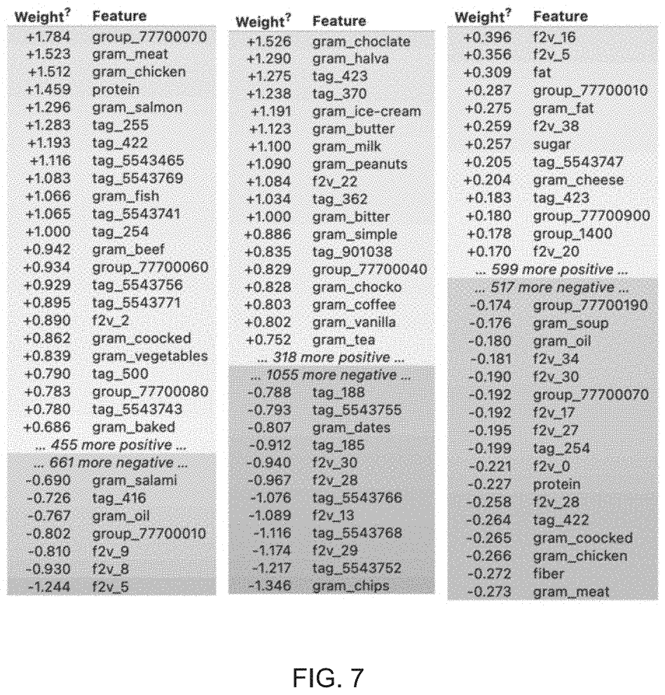

FIG. 7 shows feature importance for the is_heavy_meal_classifier (left) the is_snack classifier (middle), and the is_light classifier. Due to the good quality of the food and meals vectors trained as explained in Example 2, high results were achieved in all the classification tasks (f1 score>0.9, Table 1). Each model was measured using f1 score in order to take into consideration both precision and recall.

TABLE 1

| | is heavy meal | is snack meal | is light meal |
|---|---|---|---|
| | classifier | classifier | classifier |
| F1 score | 0.90 | 0.93 | 0.90 |
| Train set (pos/neg) | 400 (105/295) | 1200 (161/1039) | 400 (231/169) |
| Features | 1145 | 1402 | 1145 |

### EXAMPLE 4

Following is a description of a machine learning food ranking procedure suitable for the present embodiments. The ranking procedure provides a layer in the method in which it is possible to tune the recommendation according to errors and/or based on feedback from users that is preferably provided in an on-going manner. The ranking procedure is designed according to some embodiments of the present invention to rank the same recommendation list differently to different subjects. For example if subjects i and j are eating the same meal and they both get the same recommendations (r₁, r₂. . . , rₙ), the ranking model can re-rank the food items in a different order for each user and thus create a more personalized experience. The ranking procedure provides an advantage over existing recommendation systems.

Method

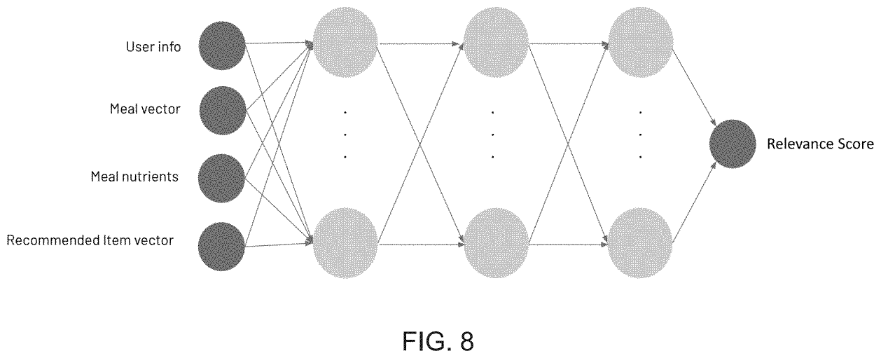

The ranking procedure of this example is a deep neural network based ranking model. The advantage of using deep neural network is because it is expected to have an increasing number of training entries, for example, by collecting usage data from the users. Deep neural network models have shown great results in past work when it comes to learning from huge datasets as oppose to traditional machine learning models which usually reach a plateau after a certain number of train entries. FIG. 8 illustrates the architecture of the ranking procedure of the present embodiments. The neural network includes 3 hidden layers of size 128, 64, 32, a dropout rate of 0.25 and a learning rate of 0.001. The TF ranking library [8] was used in order to prepare and train the network.

Model Input

The model received as input four main parameters:

1) A meal vector as described in Example 2

2) The macronutrients values of the meal (carbohydrates, fats, dietary-fibers, calories, protein, sugar, water, weight)

3) The recommended database vector

4) The subject descriptor features

Model Output

The model uses a ReLU activation function in the last layer so the output of the model is a positive number representing the strength of the relation between the input meal and the input recommended item. During train time, a zero value was provided when the meal and the recommended item are not related, and a value of 1 when they match to each other.

Results

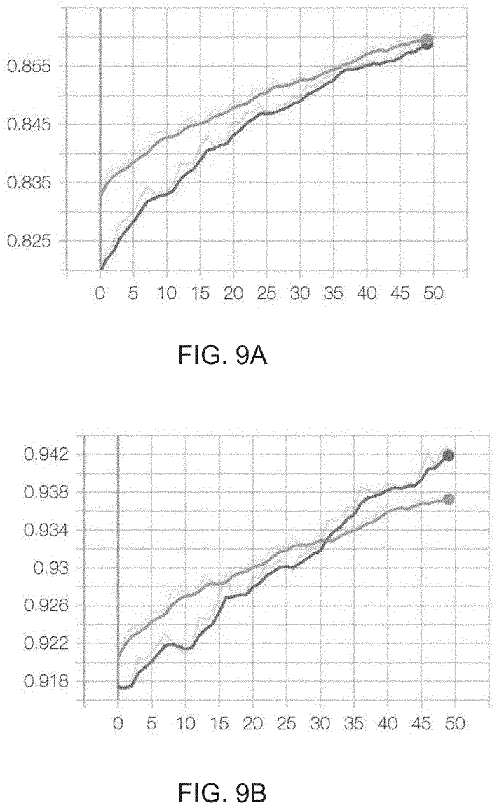

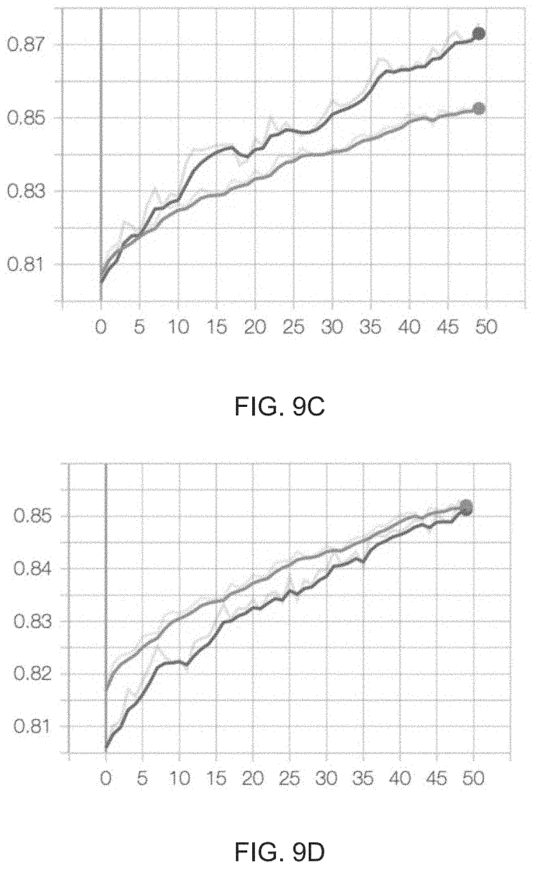

FIGS. 9A-D provides the results of the US ranking model as evaluated it using two well-known metrics used widely in the information retrieval domain:

1) NDCG@k

2) Mean Average Precision—MAP is the mean of AP over all the queries

$$AP = {\sum }_{n} ( {R}_{n} - {R}_{n - 1} ) {P}_{n}$$

FIG. 9A shows the ranking model mean average precision results as a function of the epoch, and FIGS. 9B-C the ranking model NDCG@k value as a function of the epoch. FIG. 9C shows the NDCG values of the training set and the validation set, and FIG. 9D shows the MAP values of the training set and the validation set. The abscissa of FIGS. 9C and 9D shows the epochs.

### EXAMPLE 5

A set of n=1000 beta subjects were analyzed for 2 weeks and their usage of the GUI was tracked, together with their interactions with the recommendations they were provided with by the server computer. After a 2 weeks period, more subjects were analyzed. The main measured metrics were: 1) active users engagement. 2) recommendations precision. 3) meals scores improvements. 4) selected items ranking and MRR (mean reciprocal rank). The overall recommendations results are provided in Table 2.

TABLE 2

| Metric | Value |
|---|---|
| Recommendation Precision | 88.5% |
| MRR of selected items | 0.71 |
| MRR of disliked items | 0.58 |

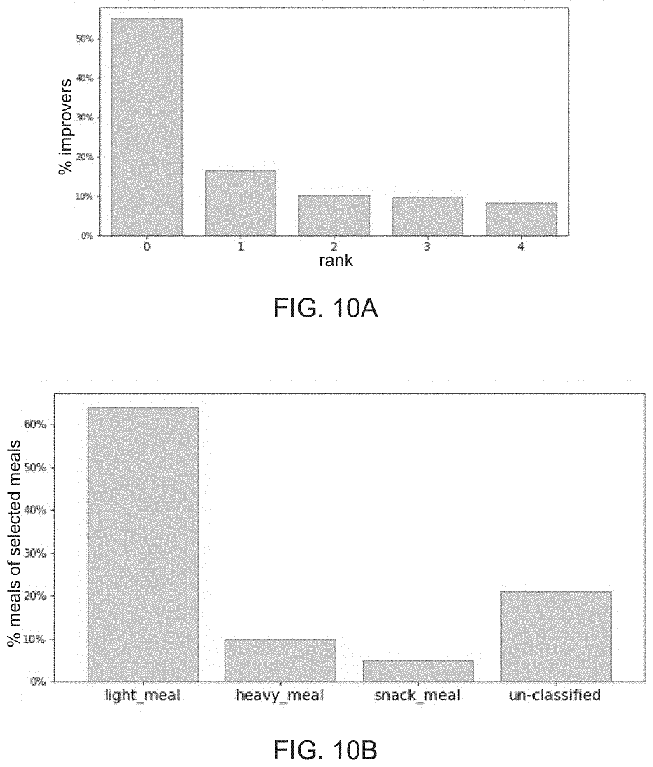

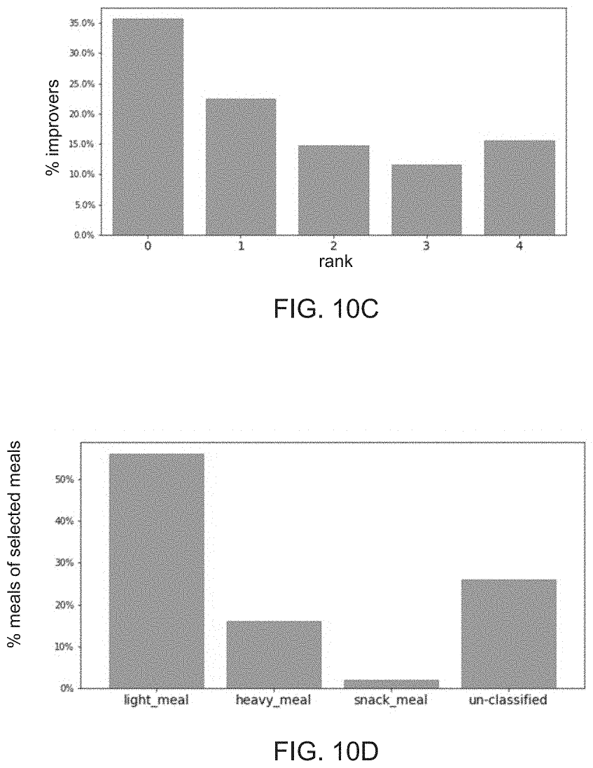

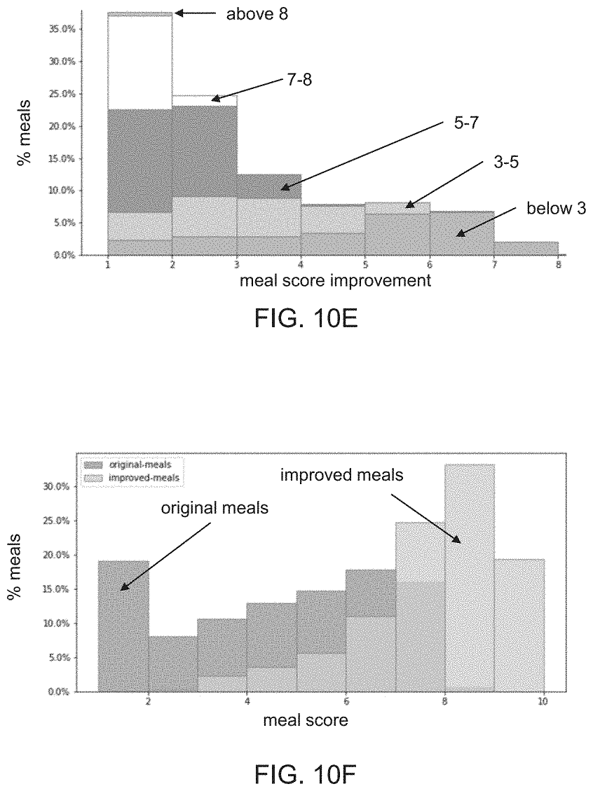

FIGS. 10A and 10B are histograms showing percentage of selected recommendations by their rank (FIG. 10A) and meal type (FIG. 10B), FIGS. 10C and 10D are histograms showing percentage of disliked recommendations by their rank (FIG. 10C) and meal type (FIG. 10D), FIG. 10E is a gray-scale coded histogram, showing percentage of meals at improvement scores bin, and FIG. 1OF is a gray-scale coded histogram, showing meals scores before and after using the recommendations.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

REFERENCES
Buysschaert et al, 2015Buysschaert M, Medina JL, Bergman M, Shah A, Lonier J. Prediabetes and associated disorders. Endocrine. 2015 Mar;48(2):371-93. doi: 10.1007/s12020-014-0436-2. Epub 2014 Oct 8. PMID: 25294012.
Markus et al, 2017Markus MRP, Ittermann T, Baumeister SE, Huth C, Thorand B, Herder C, Roden M, Siewert-Markus U, Rathmann W, Koenig W, Dörr M, Volzke H, Schipf S, Meisinger C. Prediabetes is associated with microalbuminuria, reduced kidney function and chronic kidney disease in the general population: The KORA (Cooperative Health Research in the Augsburg Region) F4-Study. Nutr Metab Cardiovasc Dis. 2018 Mar;28(3):234-242. doi: 10.1016/j.numecd.2017.12.005. Epub 2017 Dec 22. PMID: 29337019.
O'Keefe et al, 20070O'Keefe JH, Bell DS. Postprandial hyperglycemia/hyperlipidemia (postprandial dysmetabolism) is a cardiovascular risk factor. Am J Cardiol. 2007 Sep 1;100(5):899-904. doi: 10.1016/j.amjcard.2007.03.107. Epub 2007 Jun 26. PMID: 17719342.
Menke et al, 2015Menke A, Casagrande S, Geiss L, Cowie CC. Prevalence of and trends in diabetes among adults in the United States, 1988-2012. JAMA. 2015;314(10):1021-1029. doi:10.1001/jama.2015.10029
Zeevi at al, 2015Personalized Nutrition by Prediction of Glycemic Responses
Schnabel et al, 2015 Evaluation methods for unsupervised word embeddings www(dot)aclweb(dot)org/anthology/D15-1036(dot)pdf
Mikolov et al, 2013Efficient Estimation of Word Representations in Vector Space arxiv(dot)org/abs/1301.3781
Pasumarthi et al, 2019 TF-Ranking: Scalable TensorFlow Library for Learning-to-Rank arxiv(dot)org/abs/1812.00073
Zhang et al, 2019 Deep Learning based Recommender System: A Survey and New Perspectives arxiv(dot)org/abs/1707.07435

What is claimed is:

1. A method of providing a subject with a personalized diet, comprising:
receiving from a graphical user interface (GUI) at a remote location a list of food items defining a meal;
obtaining a set of subject descriptor features specific to the subject;
constructing a meal vector based on said list;
accessing a database storing a plurality of database vectors, each defining a food item, calculating similarities between said meal vector and said database vectors, selecting, based on said similarities and said set of features, at least one database vector, and, based on said selection, transmitting to the remote location identification of at least one food item for displaying said identification on said GUI as an addition to said meal.

2. The method of claim 1, comprising representing each food item of said meal as a food vector, thereby providing a list of food vectors, and wherein said constructing said meal vector is based on said list of food vectors.

3. The method of claim 2, wherein said constructing said meal vector comprise averaging over said list of food vectors.

4. The method of claim 2, wherein said representing each food item of said meal as a food vector is by a look-up table associating food items with food vectors.

5. The method of claim 1, comprising obtaining a cohort to which the subject belongs, wherein said selecting is based also on said cohort.

6. The method of claim 2, comprising obtaining a cohort to which the subject belongs, wherein said representing is based also on said cohort.

7. The method of claim 6, comprising accessing a computer-readable medium storing a library of look-up tables, each corresponding to a different cohort and each associating food items with food vectors, and selecting from said library a look-up table corresponding to said obtained cohort, wherein said representing is by said selected look-up table.

8. The method according to claim 1, comprising accessing a computer-readable medium storing a machine learning procedure trained to classify meal vectors into one of a plurality of predefined meal classifications, feeding said machine learning procedure with said constructed meal vector, and receiving from said procedure an output indicative of a classification of said constructed meal vector, wherein said selecting is based also on said classification.

9. The method of claim 8, wherein said plurality of predefined meal classifications comprises a first meal classification defined as a primary meal, a second meal classification defined as a secondary meal, and a third meal classification defined as a snack.

10. The method of claim 8, comprising obtaining a cohort to which the subject belongs, wherein said selecting is based also on said cohort.

11. The method according to claim 10, wherein said computer-readable medium stores a library of machine learning procedures each corresponding to a different cohort, wherein the method comprises selecting from said library a machine learning procedure corresponding to said obtained cohort, and wherein said feeding and said receiving is with respect to said selected procedure.

12. The method according to claim 1, wherein said selecting comprises selecting k database vectors which are most similar to said meal vector, k being a positive integer, and wherein the method comprises accessing a computer-readable medium storing a machine learning procedure trained to classify meal vectors into one of a plurality of predefined meal classifications, feeding said machine learning procedure with said constructed meal vector, receiving from said procedure an output indicative of a classification of said constructed meal vector, and filtering said k database vectors based on said classification.

13. The method of claim 12, comprising obtaining a cohort to which the subject belongs, wherein said selecting is based also on said cohort.

14. The method according to claim 10, wherein said computer-readable medium stores a library of machine learning procedures each corresponding to a different cohort, wherein the method comprises selecting from said library a machine learning procedure corresponding to said obtained cohort, and wherein said feeding and said receiving is with respect to said selected procedure.

15. The method according to claim 1, wherein a plurality of database vectors are selected, and the method comprises accessing a computer-readable medium storing a machine learning procedure trained to rank food items based on subject descriptor features, feeding said machine learning procedure with said set of subject descriptor features and each of said database vectors, and receiving from said procedure an output indicative of rankings of said database vectors for the subject, wherein said transmission of said identification of said at least one food item is based on said rankings.

16. The method according to claim 15, comprising decomposing each food item of said meal into a plurality of food descriptors, and feeding said machine learning procedure also with said plurality of food descriptors.

17. The method according to claim 16, wherein at least one of said food descriptors is selected from the group consisting of a carbohydrate content of said food item, a fat content of said food item, a dietary-fiber content of said food item, a caloric value content of said food item, a protein content of said food item, a sugar content of said food item, a water content of said food item, and a total weight of said food item.

18. The method according to claim 1, wherein a plurality of database vectors are selected, and the method comprises accessing a computer-readable medium storing a machine learning procedure trained to predict responses to food items based on subject descriptor features, feeding said machine learning procedure with said set of subject descriptor features and each of said database vectors, and receiving from said procedure an output indicative of predictions of responses of the subject to food items described by said database vectors, wherein said transmission of said identification of said at least one food item is based on said predictions.

19. A computer software product, comprising a computer-readable medium in which program instructions are stored, which instructions, when read by a data processor, cause the data processor to execute the method according to claim 1.

20. A server system for providing a subject with a personalized diet, the server system comprising:
a transceiver arranged to receive and transmit information on a communication network; and
a processor arranged to communicate with the transceiver, and execute the method according to claim 1.
